(19) United States
(12) Patent Application Publication
(10) Pub. No.: US 2017/0174522 A1
(43) Pub. Date: Jun. 22, 2017

(54) NITROGEN-DOPED GRAPHENE ASSEMBLY AND METHOD OF PREPARING THE SAME

(71) Applicants: Ho Seok PARK, Yongin-si (KR); Hyeryeon YU, Daejeon (KR); Haewon CHEONG, Sejong (KR)

(72) Inventors: Ho Seok PARK, Yongin-si (KR); Hyeryeon YU, Daejeon (KR); Haewon CHEONG, Sejong (KR)

(73) Assignee: AGENCY FOR DEFENSE DEVELOPMENT, Daejeon (KR)

(21) Appl. No.: 15/203,915

(22) Filed: Jul. 7, 2016

(30) Foreign Application Priority Data
Dec. 16, 2015 (KR) 10-2015-0180456

(51) Int. Cl. C01B 31/04; H01M 4/583; C07D 401/14

(52) U.S. Cl. CPC C01B 31/0484; C07D 401/14; H01M 4/583

(57) ABSTRACT

The present invention relates to a nitrogen-doped 3-D porous graphene assembly and a method of preparing the same. The present invention provides a method of preparing a nitrogen-doped graphene assembly, the method including the steps of mixing a graphene oxide solution with a melamine solution, freezing the mixed solution of graphene oxide and melamine, drying the frozen solution in a frozen state to prepare a graphene assembly, and heating the graphene assembly at a predetermined temperature under the argon atmosphere for a predetermined to time, in which a mass ratio of the graphene oxide and the melamine in the mixed solution is 19:1 to 4:1. According to the present invention, a uniformly nitrogen-doped 3-D porous graphene assembly may be synthesized.

### CROSS-REFERENCE TO RELATED APPLICATION

Pursuant to 35 U.S.C. §119(a), this application claims the benefit of earlier filing date and right of priority to Korean Application No. 10-2015-0180456, filed on Dec. 16, 2015, the contents of which are incorporated by reference herein in its entirety.

### BACKGROUND OF THE INVENTION

1. Field of the Invention

The present invention relates to a nitrogen-doped 3-D porous graphene assembly and a method of preparing the same.

2. Background of the Invention

Graphitic carbon materials including fullerene, carbon nanotubes, and graphene as nano-materials composed only of carbon atoms have received much attention from the academic world and industries for their excellent electrical properties, and physical and chemical stability.

In particular, graphene is a material which has come into the spotlight as an epoch-making new material due to the very high specific area compared with the volume, excellent electric conductivity, and physical and chemical stability.

Meanwhile, studies have been experimentally conducted on doping into carbon lattices for recent few years. Doping is a process which may improve electrical properties such as sheet-resistance and charge mobility of graphene When previous studies related to doping are reviewed, there are largely two methods, and examples thereof include a method in which doping is performed while graphene is synthesized, a method of modifying the material after graphene is synthesized, and the like.

However, for the existing methods of performing doping, the existing structural body may not be maintained using a separate doping device such as a gas tube or a deposition apparatus, and an extremely small amount of graphene may not be 2-dimensionally obtained.

### SUMMARY OF THE INVENTION

Therefore, an aspect of the detailed description is to provide a method which may uniformly dope a large amount of graphene while maintaining the is advantages of a 3-D porous structural body.

Further, an object of the present invention is to provide a method of preparing a 3-D graphene assembly which may quantitatively control a nitrogen element doping, and a nitrogen-doped 3-D graphene assembly, which is prepared by the method.

To achieve these and other advantages and in accordance with the purpose of this specification, as embodied and broadly described herein, there is provided a method of preparing a nitrogen-doped graphene assembly, the method including the steps of mixing a graphene oxide solution with a melamine solution, freezing the mixed solution of graphene oxide and melamine, drying the frozen solution in a frozen state to prepare a graphene assembly, and heating the graphene assembly at a predetermined temperature under the argon atmosphere for a predetermined time, in which a mass ratio of the graphene oxide and the melamine in the mixed solution is 19:1 to 4:1.

In an Example, in the mixing of the graphene oxide solution with the melamine solution, a phytic acid solution at a predetermined concentration may be additionally mixed.

In an Example, in the freezing of the mixed solution of graphene oxide and melamine, the mixed solution of graphene oxide and melamine may be frozen by adding liquid nitrogen to the mixed solution of graphene oxide and melamine.

In an Example, the predetermined temperature is characterized to be 750 to 850° C., and the predetermined time may be 1 to 2 hours.

Further, the present invention provides a nitrogen-doped graphene assembly prepared by the above-described preparation method.

In an Example, the graphene assembly may include at least one of pyridinic, pyrrolic, graphitic, and oxide pyridinic structures.

Further scope of applicability of the present application will become more apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from the detailed description.

### BRIEF DESCRIPTION OF THE DRAWINGS

The accompanying drawings, which are included to provide a further understanding of the invention and are incorporated in and constitute a part of this specification, illustrate exemplary embodiments and together with the description serve to explain the principles of the invention.

In the drawings:

FIG. 1 is a flowchart illustrating a method of preparing a nitrogen-doped graphene assembly according to an Example of the present invention;

FIG. 2 is a transmission electron microscope photograph of a nitrogen-doped graphene assembly according to an Example of the present invention;

FIG. 3 is a conceptual view for illustrating a structure of a nitrogen-doped to graphene assembly according to an Example of the present invention;

FIGS. 4a to 6b are XPS patterns of a nitrogen-doped graphene assembly according to an Example of the present invention;

FIG. 7 is a graph illustrating electrochemical characteristics of a nitrogen-undoped graphene assembly;

FIG. 8 is a graph illustrating electrochemical characteristics of a nitrogen-doped graphene assembly according to an Example of the present invention; and

FIG. 9 is a graph illustrating the change in specific capacity of the nitrogen-undoped graphene assembly and the nitrogen-doped graphene assembly according to an Example of the present invention.

### DETAILED DESCRIPTION OF THE INVENTION

Hereinafter, an Example according to the present invention will be specifically described. In the present specification, like reference numbers are used to designate like constituents even though they are in different Examples, and the description thereof will be substituted with the initial description. Singular expressions used in the present specification include plural expressions unless they have definitely opposite meanings in the context.

The present invention provides a method of preparing a nitrogen-doped graphene assembly. Hereinafter, the present invention will be specifically described with reference to the accompanying drawings.

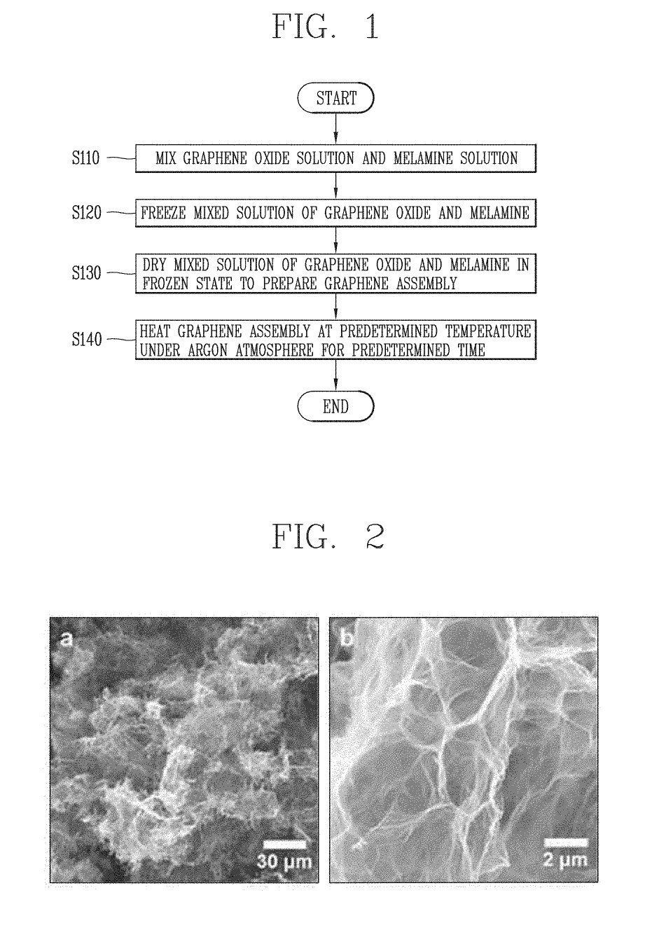

FIG. 1 is a flowchart illustrating a method of preparing a nitrogen-doped graphene assembly according to an Example of the present invention.

In order to prepare a nitrogen-doped graphene assembly, a step of mixing a to graphene oxide solution with a melamine solution (S110) is carried out.

Graphene has a honeycomb structure and is structurally very stable as a 2-D material formed of carbon atoms.

Graphene may be obtained from graphite, and graphene may be peeled off from graphite. In order to peel off graphene from graphite, a chemical method is may be utilized. Specifically, when functional groups are introduced into graphite through an oxidation reaction, a graphene oxide is easily dispersed in the solution. Subsequently, graphene may be obtained by reducing the graphene oxide, if necessary. In the present invention, the graphene oxide is used in order to prepare a graphene assembly.

Meanwhile, melamine may be represented by Chemical Formula 1.

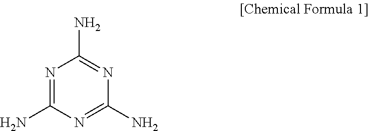

Melamine serves as a nitrogen supply source which supplies nitrogen to graphene, and in a mixed solution of graphene oxide and melamine, the structure of the graphene assembly and the nitrogen element ratio may vary according to the mass ratio of the graphene oxide and melamine.

The mass ratio of the graphene oxide and melamine may be 19:1 to 4:1.

Meanwhile, melamine is present in a solid state at room temperature, and thus may be allowed to be mixed with the graphene oxide after being dispersed in distilled water at high temperature.

When the graphene oxide is mixed with melamine, melamine is attached to the graphene oxide. In this case, the graphene oxide and melamine are not chemically reacted.

Meanwhile, phytic acid at a predetermined concentration may be additionally mixed with the mixed solution of graphene oxide and melamine. Through this, the graphene assembly may be doped with phosphorous together with nitrogen atoms.

Next, a step of freezing the mixed solution of graphene oxide and melamine (S120) is carried out.

When the mixed solution of graphene oxide and melamine is frozen, the graphene oxides are aggregated with each other to form a 3-D graphene structural body. In this case, the 3-D graphene structural body may have a porous structure.

In order to freeze the mixed solution of graphene oxide and melamine, s liquid nitrogen may be used.

Next, a step of drying the mixed solution of graphene oxide and melamine in a frozen state to prepare a graphene assembly (S130) is carried out.

When the mixed solution is dried in a frozen state, it is possible to prevent destruction of a 3-D structure which the graphene structural body forms.

Finally, a step of heating the graphene assembly at a predetermined temperature under an argon atmosphere for a predetermined time (S140) is carried out.

The predetermined temperature may be 750 to 850° C., and the predetermined time may be 1 to 2 hours.

The above-described preparation method may be performed as the following example, but is not limited thereto.

### EXAMPLE

### Preparation of Nitrogen-Doped Graphene Assembly

16 mg of melamine was put into 20 ml of distilled water, and the mixture was dispersed at 80° C. for 20 minutes. A dispersed aqueous solution including 80 mg of a 2-D graphene oxide was homogenously mixed with the melamine dispersed aqueous solution. The mixed solution was frozen using liquid nitrogen, and then freeze-dried to prepare a 3-D porous graphene assembly.

The 3-D porous graphene assembly was heated at 800° C. under the argon atmosphere for 1 hour. In this case, the flow rate of argon was 50 cc/min, and the heating rate was 5° C./min.

The graphene assembly was cooled to room temperature under the argon atmosphere in the same condition as during the heating.

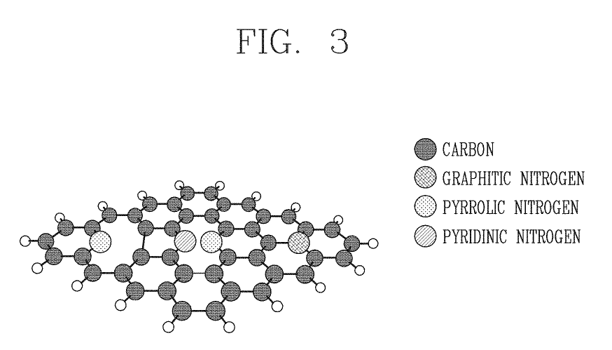

FIG. 2 is a transmission electron microscope photograph of a nitrogen-doped graphene assembly according to an Example of the present invention. As in FIG. 2, it can be confirmed that the 3-D porous graphene assembly is assembled such that graphene is 3-dimensionally connected to each other, and there is a plurality of pores on the surface and inside thereof.

Hereinafter, the structure of the nitrogen-doped graphene assembly prepared by the above-described method will be specifically described.

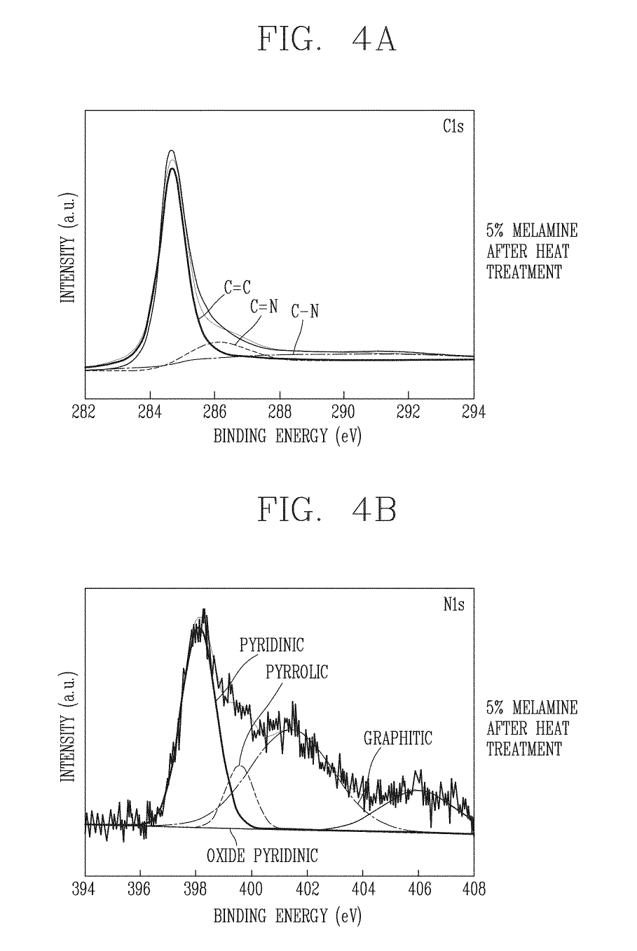

FIG. 3 is a conceptual view for illustrating a structure of a nitrogen-doped graphene assembly according to an Example of the present invention.

The structure of a cross-section of the nitrogen-doped graphene assembly of the present invention may be represented as in FIG. 3. Specifically, the nitrogen-doped graphene assembly of the present invention may include at least is one of pyridinic, pyrrolic, graphitic, and oxide pyridinic structures. Here, the pyridinic, pyrrolic, graphitic, and oxide pyridinic structures may have a structure similar to pyridine, pyrrole, quaternary amine, and oxide pyridine, respectively as in FIG. 3. Pyridine, pyrrole, quaternary amine, and oxide pyridine may be represented by Chemical Formulae 2 to 5 in this order.

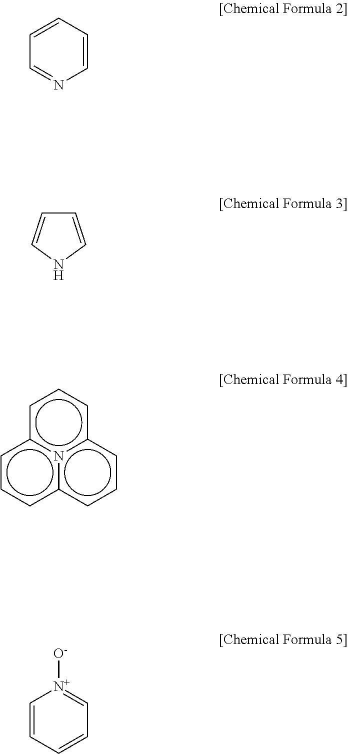

Meanwhile, nitrogen elements may be uniformly distributed in the graphene assembly.

Meanwhile, the nitrogen-doped graphene assembly of the present invention may include each of pyridinic, pyrrolic, graphitic, and oxide pyridinic structure at a predetermined ratio.

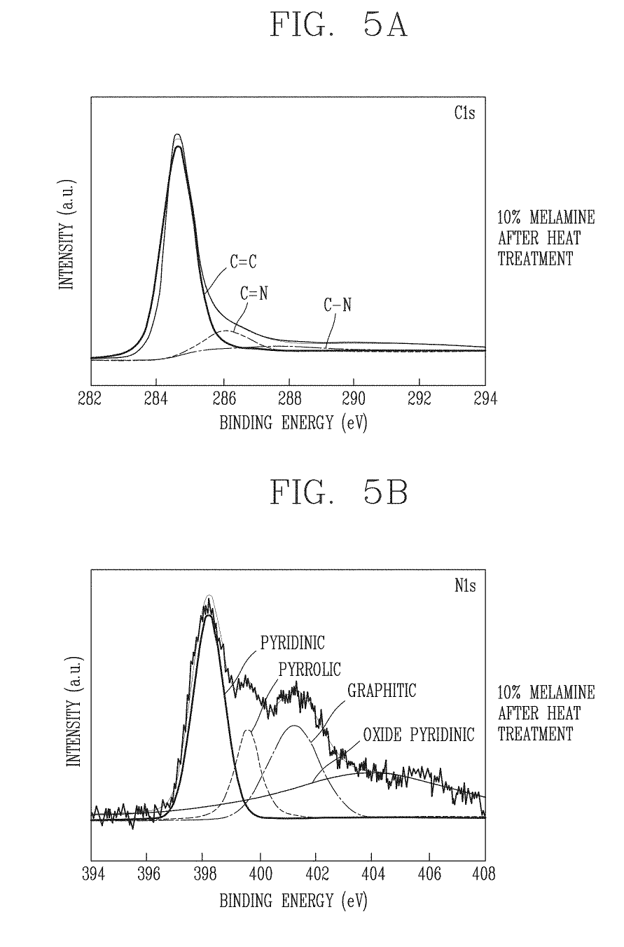

FIGS. 4a to 6b are XPS patterns of a nitrogen-doped graphene assembly according to an Example of the present invention.

According to FIGS. 4a to 6b, it can be confirmed that when the graphene assembly is prepared, the ratio of the structures may vary according to the mass ratio of the mixed melamine. Specifically, it can be confirmed that as the mass ratio of melamine is decreased, the ratio of pyridinic and pyrrolic structures is decreased, and the ratio of graphitic and oxide pyridinic structures is increased.

Meanwhile, apart from this, as the predetermined temperature is increased and the predetermined time is elongated, the ratio of pyridinic and pyrrolic structures is decreased, and the ratio of graphitic and oxide pyridinic structure is increased.

As described above, the ratio of the structures included in the graphene assembly may be adjusted by varying the mass ratio of melamine and the temperature and time of heat treatment.

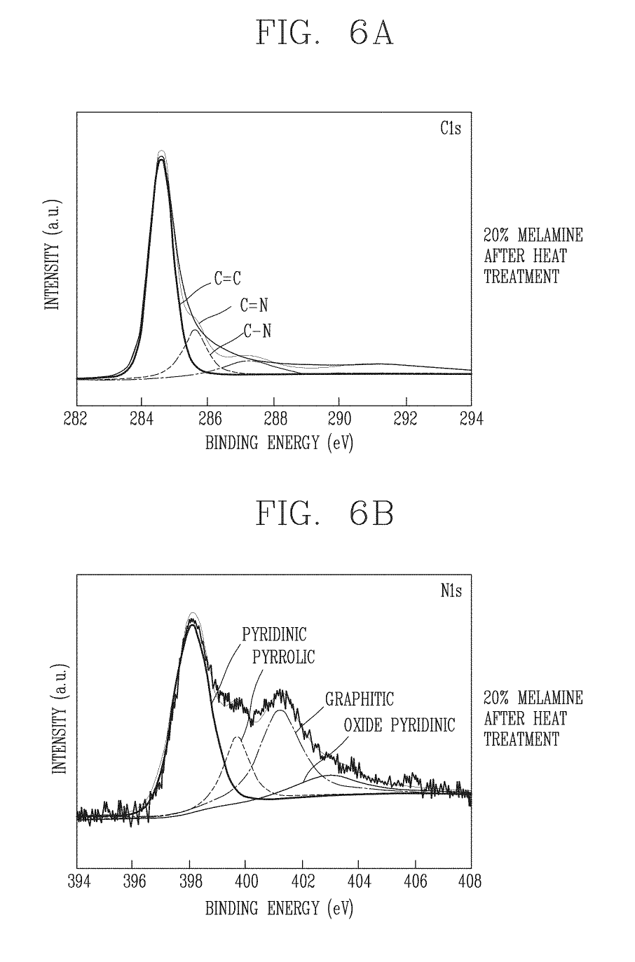

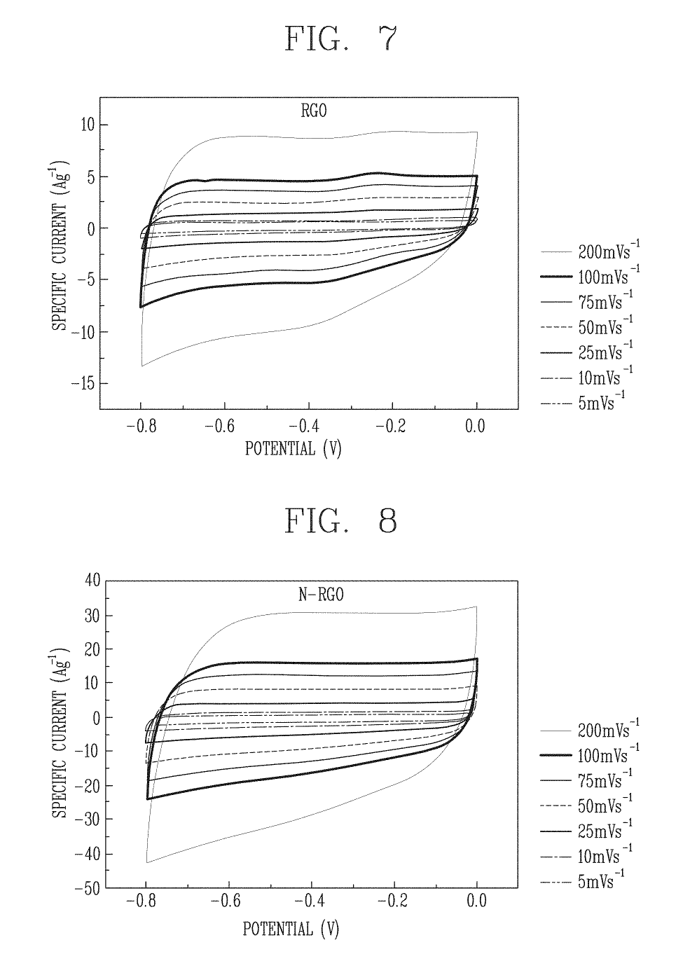

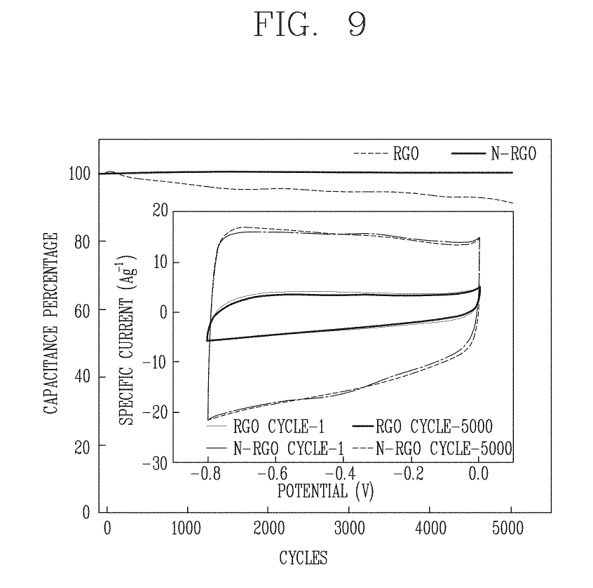

Hereinafter, electrochemical characteristics of the nitrogen-doped graphene assembly prepared by the above-described method will be specifically described. FIG. 7 is a graph illustrating electrochemical characteristics of a nitrogen-undoped graphene assembly, FIG. 8 is a graph illustrating electrochemical characteristics of a nitrogen-doped graphene assembly according to an Example of the present invention, and FIG. 9 is a graph illustrating the change in specific capacity of the nitrogen-undoped graphene assembly and the nitrogen-doped graphene assembly according to an Example of the present invention.

### Experimental Example 1

### Measurement of Specific Capacities of Graphene Assembly at Different Current Densities

The experimental conditions for measuring the specific capacities of a nitrogen-undoped graphene assembly (hereinafter, referred to as RGO) and a nitrogen-doped graphene assembly (hereinafter, referred to as NRGO) are as follows.

3-electrode system: R.E:Ag/AgCl, C.E:Platinum, W.E:Sample slurry on Ti plate

Electrolyte: 6M KOH Potential Window: −0.8 to 0

Slurry Preparation: Grinding method (80% Sample, 20% PVDF & NMP)

The results of analyzing electrochemical characteristics of the graphene assembly according to the experimental conditions are the same as each other in FIGS. 7 and 8.

The specific capacity of the NRGO is 214.6 F/g, which is higher than that of the RGO (72.7 F/g).

### Experimental Example 2

### Analysis of Cycle Stability of Graphene Assembly

The measurement of the specific capacity in Experimental Example 1 was repeated 5,000 cycles, and the specific capacity of the graphene assembly was observed.

As in FIG. 9, the specific capacity of the RGO was decreased to 91% after 500 cycles. In contrast, the specific capacity of the NRGO was maintained to 100% even after 5,000 cycles.

As the present features may be embodied in several forms without departing from the characteristics thereof, it should also be understood that the above-described embodiments are not limited by any of the details of the foregoing description, unless otherwise specified, but rather should be construed broadly within its scope as defined in the appended claims, and therefore all changes and modifications that fall within the metes and bounds of the claims, or equivalents of such metes and bounds are therefore intended to be embraced by the appended claims.

As the present features may be embodied in several forms without departing from the characteristics thereof, it should also be understood that the above-described embodiments are not limited by any of the details of the foregoing description, unless otherwise specified, but rather should be construed broadly within its scope as defined in the appended claims, and therefore all changes and modifications that fall within the metes and bounds of the claims, or equivalents of such metes and bounds are therefore intended to be embraced by the appended claims.

1. A method of preparing a nitrogen-doped graphene assembly, the method comprising: the steps of
mixing a graphene oxide solution with a melamine solution;
freezing the mixed solution of graphene oxide and melamine;
drying the frozen solution in a frozen state to prepare a graphene assembly; and
heating the graphene assembly at a predetermined temperature under an argon atmosphere for a predetermined time,
wherein a mass ratio of the graphene oxide and the melamine in the mixed solution is 19:1 to 4:1.

2. The method of claim 1, wherein in the mixing of the graphene oxide solution with the melamine solution,
a phytic acid solution at a predetermined concentration is additionally mixed.

3. The method of claim 1, wherein in the freezing of the mixed solution of graphene oxide and melamine,
the mixed solution of graphene oxide and melamine is frozen by adding liquid nitrogen to the mixed solution of graphene oxide and melamine.

4. The method of claim 1, wherein the predetermined temperature is 750 to 850° C., and the predetermined time is 1 to 2 hours.

5. A nitrogen-doped graphene assembly prepared by the preparation method of claim 1.

6. The graphene assembly of claim 5, wherein the graphene assembly comprises at least one of pyridinic, pyrrolic, graphitic, and oxide pyridinic structures.
